(19) United States
(12) Patent Application Publication
(10) Pub. No.: US 2015/0191688 A1
(43) Pub. Date: Jul. 9, 2015

(54) MULTIWELL PLATES COMPRISING NANOWIRES

(71) Applicant: Presodemt and Fellows of Harvard College, Cambridge, MA (US)

(72) Inventors: Hongkun Park, Lexington, MA (US); Alexander K. Shalek, Cambridge, MA (US); Ruihua Ding, Allston, MA (US); Joseph Park, Belmont, MA (US)

(21) Appl. No.: 14/422,565

(22) PCT Filed: Mar. 15, 2013
(86) PCT No.: PCT/US2013/032512

§ 371 Date: Feb. 19, 2015

Related U.S. Application Data
(60) Provisional application No. 61/692,017, filed on Aug. 22, 2012.

(51) Int. Cl. C12M 1/32; C12M 1/12; C12N 5/00; C12N 1/20

(52) U.S. Cl. CPC C12M 23/12; C12N 1/20; C12M 25/00; C12N 5/0068

(57) ABSTRACT

The present invention generally relates to nanowires and, in particular, to multiwell plates comprising nanowires, including systems and methods of making the same. Such multiwell plates can, in some cases, be used in automated equipment or high-throughput applications. For example, a plurality of cells may be placed in at least some of the wells of the multiwell plate, and one or more nanowires may be inserted into at least some of the cells within the wells of the multiwell plate. In some cases, one or more of the nanowires may have coated thereon a biological effector. The cells in each of the wells may be identical or different, and/or the biological effector may the same or different. Such multiwell plates may be used, for example, to test a biological effector against a variety of cell types, or to test a variety of biological effectors against a one or more cell types, or the like.

### RELATED APPLICATIONS

This application claims the benefit of U.S. Provisional Patent Application Ser. No. 61/692,017, filed Aug. 22, 2012, entitled “Fabrication of Nanowire Arrays,” by Hongkun Park, et al., incorporated herein by reference.

### GOVERNMENT FUNDING

Research leading to various aspects of the present invention was sponsored, at least in part, by the National Institutes of Health, Grant No. 8DP1DA035083-05. The U.S. Government has certain rights in the invention.

### FIELD

The present invention generally relates to nanowires and, in particular, to multiwell plates comprising nanowires.

### BACKGROUND

Nanowires (NWs) provide a powerful new system for delivering biological effectors directly into a wide variety of cells. However, due to their size, typically on the order of nanometers, it is difficult to expose arrays of nanowires and cells to different conditions. Accordingly, improvements are needed.

### SUMMARY

The present invention generally relates to nanowires and, in particular, to multiwell plates comprising nanowires. The subject matter of the present invention involves, in some cases, interrelated products, alternative solutions to a particular problem, and/or a plurality of different uses of one or more systems and/or articles.

In one aspect, the present invention is generally directed to an article comprising a bottomless multiwell plate, and a substrate comprising a plurality of upstanding nanowires immobilized to the multiwell plate.

In another aspect, the present invention is generally directed to a method. In one set of embodiments, the method comprises immobilizing a substrate comprising a plurality of upstanding nanowires to a bottomless multiwell plate. In another set of embodiments, the method comprises placing a plurality of cells in a plurality of wells in a multiwell plate, where at least one of the wells comprises a plurality of upstanding nanowires.

The method, in still another set of embodiments, comprises placing at least 10 distinct cell types into at least 10 distinct wells of a multiwell plate, and inserting a plurality of nanowires coated with an identical biological effector into each of the at least 10 distinct cell types. In yet another set of embodiments, the method comprises acts of placing cells into at least 10 distinct wells of a multiwell plate, and inserting a plurality of nanowires into the cells, at least some of the nanowires at least partially coated with a biological effector, wherein in each of the 10 distinct wells, a different biological effector is inserted into the cells in the respective wells.

Other advantages and novel features of the present invention will become apparent from the following detailed description of various non-limiting embodiments of the invention when considered in conjunction with the accompanying FIGURE. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

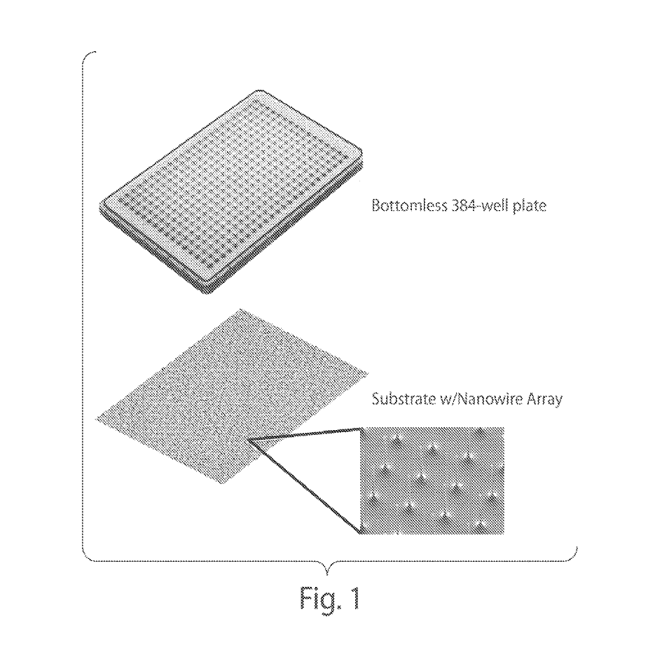

Non-limiting embodiments of the present invention will be described by way of example with reference to the accompanying FIGURE, which are schematic and are not intended to be drawn to scale. In the FIGURE, each identical or nearly identical component illustrated is typically represented by a single numeral. For purposes of clarity, not every component is labeled in every FIGURE, nor is every component of each embodiment of the invention shown where illustration is not necessary to allow those of ordinary skill in the art to understand the invention. In the FIGURE: FIG. 1 provides a schematic depiction of the components of the multiwell nanowire array plate, in accordance with one embodiment of the invention.

### DETAILED DESCRIPTION

The present invention generally relates to nanowires and, in particular, to multiwell plates comprising nanowires, including systems and methods of making the same. Such multiwell plates can, in some cases, be used in automated equipment or high-throughput applications. For example, a plurality of cells may be placed in at least some of the wells of the multiwell plate, and one or more nanowires may be inserted into at least some of the cells within the wells of the multiwell plate. In some cases, one or more of the nanowires may have coated thereon a biological effector. The cells in each of the wells may be identical or different, and/or the biological effector may the same or different. Such multiwell plates may be used, for example, to test a biological effector against a variety of cell types, or to test a variety of biological effectors against a one or more cell types, or the like.

In one aspect, the present invention is generally directed to multiwell plates comprising nanowires, as discussed below. The multiwell plates may be of any size. However, in certain embodiments, the multiwell plate has the dimensions of a microwell plate, e.g., having standard dimensions (about 5 inches×about 3.33 inches, or about 128 mm×86 mm) and/or standard numbers of wells therein. For example, there may be 6, 24, 48, 96, 384, 1536 or 3456 wells present in the multiwell plate. Multiwell plates may be fabricated from any suitable material, e.g., polystyrene, polypropylene, polycarbonate, cyclo-olefins, or the like. Microwell plates can be made by injection molding, casting, machining, laser cutting, or vacuum sheet forming one or more resins, and can be made from transparent or opaque materials. Many such microwell plates are commercially available.

In one set of embodiments, the multiwell plate is prepared by immobilizing a bottomless multiwell plate with a substrate comprising a plurality of upstanding nanowires. For example, the bottomless multiwell plate may be a commercially available bottomless microwell plate, e.g., a bottomless 384-well microwell plate, e.g., as is shown in FIG. 1. The substrate and the nanowires may comprise semiconductor materials such as silicon, or other materials as described herein.

In some embodiments, the multiwell plate and the substrate may be immobilized with respect to each other by the use of a suitable adhesive. Non-limiting examples of adhesives include acrylic adhesives, pressure-sensitive adhesives, silicone adhesives (e.g., UV curable silicones or RTV silicones), biocompatible adhesives, epoxies, or the like. Non-limiting examples of biocompatible glues include, but are not limited to, Master Bond EP42HT-2ND-2MED BLACK and Master Bond EP42HT-2 CLEAR (Master Bond). The adhesive, in some cases, may be a permanent adhesive. Many such adhesives can be obtained commercially from companies such as 3M, Loctite, or Adhesives Research.

The multiwell plate and the substrate may be directly immobilized to each other, and/or there may be other materials positioned between the multiwell plate and the substrate, for example, one or more gaskets (e.g., comprising silicone, rubber, neoprene, nitrile rubber, fiberglass, polytetrafluoroethylene, etc.). In some cases, these materials may be dimensioned and arranged to be in the same pattern as the wells (or a subset thereof) of the multiwell plate to which they are being attached. The substrate may comprise one or more upstanding nanowires. On average, the upstanding nanowires may form an angle with respect to a substrate of between about 80° and about 100°, between about 85° and about 95°, or between about 88° and about 92°. In some cases, the average angle is about 90°. As used herein, the term “nanowire” (or “NW”) refers to a material in the shape of a wire or rod having a diameter in the range of 1 nm to 1 micrometer (μm). The NWs may be formed from materials with low cytotoxicity; suitable materials include, but are not limited to, silicon, silicon oxide, silicon nitride, silicon carbide, iron oxide, aluminum oxide, iridium oxide, tungsten, stainless steel, silver, platinum, and gold. Other suitable materials include aluminum, copper, molybdenum, tantalum, titanium, nickel, tungsten, chromium, or palladium. In some embodiments, the nanowire comprises or consists essentially of a semiconductor.

Typically, a semiconductor is an element having semiconductive or semi-metallic properties (i.e., between metallic and non-metallic properties). An example of a semiconductor is silicon. Other non-limiting examples include elemental semiconductors, such as gallium, germanium, diamond (carbon), tin, selenium, tellurium, boron, or phosphorous. In other embodiments, more than one element may be present in the nanowires as the semiconductor, for example, gallium arsenide, gallium nitride, indium phosphide, cadmium selenide, etc.

The size and density of the NWs in the NW arrays may be varied; the lengths, diameters, and density of the NWs can be configured to permit adhesion and penetration of cells. In some embodiments, the length of the NWs can be 0.1-10 micrometers (μm).

In some cases, the diameter of the NWs can be 50-300 nm. In certain embodiments, the density of the NWs can be 0.05-5 NWs per micrometer² (μm²). Other examples are discussed below.

The nanowires may have any suitable length, as measured moving away from the substrate. The nanowires may have substantially the same lengths, or different lengths in some cases. For example, the nanowires may have an average length of at least about 0.1 micrometers, at least about 0.2 micrometers, at least about 0.3 micrometers, at least about 0.5 micrometers, at least about 0.7 micrometers, at least about 1 micrometer, at least about 2 micrometers, at least about 3 micrometers, at least about 5 micrometers, at least about 7 micrometers, or at least about 10 micrometers. In some cases, the nanowires may have an average length of no more than about 10 micrometers, no more than about 7 micrometers, no more than about 5 micrometers, no more than about 3 micrometers, no more than about 2 micrometers, no more than about 1 micrometer, no more than about 0.7 micrometers, no more than about 0.5 micrometers, no more than about 0.3 micrometers, no more than about 0.2 micrometers, or no more than about 0.1 micrometers. Combinations of any of these are also possible in some embodiments.

The nanowires may also have any suitable diameter, or narrowest dimension if the nanowires are not circular. The nanowires may have substantially the same diameters, or in some cases, the nanowires may have different diameters. In some cases, the nanowires may have an average diameter of at least about 10 nm, at least about 30 nm, at least about 50 nm, at least about 70 nm, at least about 100 nm, at least about 200 nm, at least about 300 nm, etc., and/or the nanowires may have an average diameter of no more than about 300 nm, no more than about 200 nm, no more than about 100 nm, no more than about 70 nm, no more than about 50 nm, no more than about 30 nm, no more than about 20 nm, or no more than about 10 nm, or any combination of these.

In addition, in some cases, the density of nanowires on the substrate, or on a region of the substrate defined by nanowires, may be at least about 0.01 nanowires per square micrometer, at least about 0.02 nanowires per square micrometer, at least about 0.03 nanowires per square micrometer, at least about 0.05 nanowires per square micrometer, at least about 0.07 nanowires per square micrometer, at least about 0.1 nanowires per square micrometer, at least about 0.2 nanowires per square micrometer, at least about 0.3 nanowires per square micrometer, at least about 0.5 nanowires per square micrometer, at least about 0.7 nanowires per square micrometer, at least about 1 nanowire per square micrometer, at least about 2 nanowires per square micrometer, at least about 3 nanowires per square micrometer, at least about 4 nanowires per square micrometer, at least about 5 nanowires per square micrometer, etc. In addition, in some embodiments, the density of nanowires on the substrate may be no more than about 10 nanowires per square micrometer, no more than about 5 nanowires per square micrometer, no more than about 4 nanowires per square micrometer, no more than about 3 nanowires per square micrometer, no more than about 2 nanowires per square micrometer, no more than about 1 nanowire per square micrometer, no more than about 0.7 nanowires per square micrometer, no more than about 0.5 nanowires per square micrometer, no more than about 0.3 nanowires per square micrometer, no more than about 0.2 nanowires per square micrometer, no more than about 0.1 nanowires per square micrometer, no more than about 0.07 nanowires per square micrometer, no more than about 0.05 nanowires per square micrometer, no more than about 0.03 nanowires per square micrometer, no more than about 0.02 nanowires per square micrometer, or no more than about 0.01 nanowires per square micrometer.

The nanowires may be regularly or irregularly spaced on the substrate. For example, the nanowires may be positioned within a rectangular grid with periodic spacing, e.g., having a periodic spacing of at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the periodic spacing may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. Combinations of these are also possible, e.g., the array may have a periodic spacing of nanowires of between about 0.01 micrometers and about 0.03 micrometers.

In some cases, the nanowires (whether regularly or irregularly spaced) may be positioned on the substrate such that the average distance between a nanowire and its nearest neighboring nanowire is at least about 0.01 micrometers, at least about 0.03 micrometers, at least about 0.05 micrometers, at least about 0.1 micrometers, at least about 0.3 micrometers, at least about 0.5 micrometers, at least about 1 micrometer, at least about 2 micrometers, at least about 3 micrometers, at least about 5 micrometers, at least about 10 micrometers, etc. In some cases, the distance may be no more than about 10 micrometers, no more than about 5 micrometers, no more than about 3 micrometers, no more than about 1 micrometer, no more than about 0.5 micrometers, no more than about 0.3 micrometers, no more than about 0.1 micrometers, no more than about 0.05 micrometers, no more than about 0.03 micrometers, no more than about 0.01 micrometers, etc. In some cases, the average distance may fall within any of these values, e.g., between about 0.5 micrometers and about 2 micrometers.

In certain aspects, the substrate may comprise more than one region of nanowires, e.g., patterned as discussed herein. For example, a pre-determined pattern of photons or electrons may be used to produce a substrate comprising a first region of nanowires and a second region of nanowires. In addition, in some cases, more than two such regions of nanowires may be produced on a substrate. For example, there may be at least 3, at least 6, at least 10, at least 15, at least 20, at least 50, or at least 100 separate regions of nanowires on a substrate. In some cases, the regions are separate from each other. Any number of nanowires may be present in a region, e.g., at least about 10, at least about 20, at least about 50, at least about 100, at least about 300, at least about 1000, etc. The nanowires may be present in any suitable configuration or array, e.g., in a rectangular or a square array.

The nanowires in a first region and a second region may be the same, or there may be one or more different characteristics between the nanowires. For example, the nanowires in the first region and the second region may have different average diameters, lengths, densities, biological effectors, or the like. If more than two regions of nanowires are present on the substrate, each of the regions may independently be the same or different.

The substrate may be formed of the same or different materials as the nanowires. For example, the substrate may comprise silicon, silicon oxide, silicon nitride, silicon carbide, iron oxide, aluminum oxide, iridium oxide, tungsten, stainless steel, silver, platinum, gold, gallium, germanium, or any other materials described herein that a nanowire may be formed from. In one embodiment, the substrate is formed from a semiconductor.

In some embodiments, arrays of NWs on a substrate may be obtained by growing NWs from a precursor material. As a non-limiting example, chemical vapor deposition (CVD) may be used to grow NWs by placing or patterning catalyst or seed particles (typically with a diameter of 1 nm to a few hundred nm) atop a substrate and adding a precursor to the catalyst or seed particles. When the particles become saturated with the precursor, NWs can begin to grow in a shape that minimizes the system's energy. By varying the precursor, substrate, catalyst/seed particles (e.g., size, density, and deposition method on the substrate), and growth conditions, NWs can be made in a variety of materials, sizes, and shapes, at sites of choice.

In certain embodiments, arrays of NWs on a substrate may be obtained by growing NWs using a top-down process that involves removing predefined structures from a supporting substrate. As a non-limiting example, the sites where NWs are to be formed may be patterned into a soft mask and subsequently etched to develop the patterned sites into three-dimensional nanowires. Methods for patterning the soft mask include, but are not limited to, photolithography and electron beam lithography. The etching step may be either wet or dry. In one set of embodiments, at least some of the NWs may be used to deliver a molecule of interest into a cell, e.g., through insertion of a NW into the cell. In certain embodiments of the invention, at least some of the NWs may undergo surface modification so that molecules of interest can be attached to them. It should be appreciated that the NWs can be complexed with various molecules according to any method known in the art. It should also be appreciated that the molecules connected to different NWs may be distinct. In some embodiments, a NW may be attached to a molecule of interest through a linker. The interaction between the linker and the NW may be covalent, electrostatic, photosensitive, or hydrolysable. As a specific non-limiting example, a silane compound may be applied to a NW with a surface layer of silicon oxide, resulting in a covalent Si—O bond. As another specific non-limiting example, a thiol compound may be applied to a NW with a surface layer of gold, resulting in a covalent Au—S bond. Examples of compounds for surface modification include, but are not limited to, aminosilanes such as (3-aminopropyl)-trimethoxysilane, (3-aminopropyl)-triethoxysilane, 3-(2-aminoethylamino)propyl-dimethoxymethylsilane, (3-aminopropyl)-diethoxy-methylsilane, [3-(2-aminoethylamino)propyl]trimethoxysilane, bis[3-(trimethoxysilyl)propyl]amine, and (11-aminoundecyl)-triethoxysilane; glycidoxysilanes such as 3-glycidoxypropyldimethylethoxysilane and 3-glycidyloxypropyl)trimethoxysilane; mercaptosilanes such as (3-mercaptopropyl)-trimethoxysilane and (11-mercaptoundecyl)-trimethoxysilane; and other silanes such as trimethoxy(octyl)silane, trichloro(propyl)silane, trimethoxyphenylsilane, trimethoxy(2-phenylethyl)silane, allyltriethoxysilane, allyltrimethoxysilane, 3-[bis(2-hydroxyethyl)amino]propyl-triethoxydilane, 3-(trichlorosilyl)propyl methacrylate, and (3-bromopropyl)trimethoxysilane. Other non-limiting examples of compounds that may be used to form the linker include poly-lysine, collagen, fibronectin, and laminin.

In addition, in various embodiments, a nanowire may be prepared for binding or coating of a suitable biological effector by activating the surface of the nanowire, silanizing at least a portion of the nanowire, and reacting a crosslinker to the silanized portions of the nanowire. Methods for activating the surface include, but are not limited to, surface oxidation, such as by plasma oxidation or acid oxidation. Non-limiting examples of suitable types of crosslinkers that are commercially available and known in the art include maleimides, histidines, haloacetyls, and pyridyldithiols. Similarly, the interaction between the linker and the molecule to be delivered can be covalent, electrostatic, photosensitive, or hydrolysable. In some embodiments, a molecule of interest attached to or coated on a NW may be a biological effector. As used herein, a “biological effector” refers to a substance that is able to modulate the expression or activity of a cellular target. It includes, but is not limited to, a small molecule, a protein (e.g., a natural protein or a fusion protein), an enzyme, an antibody (e.g., a monoclonal antibody), a nucleic acid (e.g., DNA, including linear and plasmid DNAs; RNA, including mRNA, siRNA, and microRNA), and a carbohydrate. The term “small molecule” refers to any molecule with a molecular weight below 1000 Da. Non-limiting examples of molecules that may be considered to be small molecules include synthetic compounds, drug molecules, oligosaccharides, oligonucleotides, and peptides.

The term “cellular target” refers to any component of a cell. Non-limiting examples of cellular targets include DNA, RNA, a protein, an organelle, a lipid, or the cytoskeleton of a cell. Other examples include the lysosome, mitochondria, ribosome, nucleus, or the cell membrane. In some cases, the nanowires can be used to deliver biological effectors or other suitable biomolecular cargo into a population of cells at surprisingly high efficiencies. Furthermore, such efficiencies may be achieved regardless of cell type, as the primary mode of interaction between the nanowires and the cells is physical insertion, rather than biochemical interactions (e.g., as would appear in traditional pathways such as phagocytosis, receptor-mediated endocytosis, etc.). For instance, in a population of cells on the surface of the substrate, at least about 50%, at least about 60%, at least about 70%, at least about 80%, or at least about 90% of the cells may have at least one nanowire inserted therein. In some cases, as discussed herein, the nanowires may have at least partially coated thereon one or more biological effectors. Thus, in some embodiments, biological effectors may be delivered to at least about 50%, at least about 60%, at least about 70%, at least about 80%, or at least about 90% of the cells on the substrate, e.g., via the nanowires.

In one set of embodiments, the surface of the substrate may be treated in any fashion that allows binding of cells to occur thereto. For example, the surface may be ionized and/or coated with any of a wide variety of hydrophilic and/or cytophilic materials, for example, materials having exposed carboxylic acid, alcohol, and/or amino groups. In another set of embodiments, the surface of the substrate may be reacted in such a manner as to produce carboxylic acid, alcohol, and/or amino groups on the surface. In some cases, the surface of the substrate may be coated with a biological material that promotes adhesion or binding of cells, for example, materials such as fibronectin, laminin, vitronectin, albumin, collagen, or peptides or proteins containing RGD sequences.

It should be understood that for a cell to adhere to the nanowire, a separate chemical or “glue” is not necessarily required. In some cases, sufficient nanowires may be inserted into a cell such that the cell cannot easily be removed from the nanowires (e.g., through random or ambient vibrations), and thus, the nanowires are able to remain inserted into the cells. In some cases, the cells cannot be readily removed via application of an external fluid after the nanowires have been inserted into the cells.

In some cases, merely placing or plating the cells on the nanowires is sufficient to cause at least some of the nanowires to be inserted into the cells. For example, a population of cells suspended in media may be added to the surface of the substrate containing the nanowires, and as the cells settle from being suspended in the media to the surface of the substrate, at least some of the cells may encounter nanowires, which may (at least in some cases) become inserted into the cells.

Thus, certain aspects of the invention are directed to multiwell plates comprising a plurality of upstanding nanowires within at least some of the wells of the multiwell plates. In some embodiments, at least about 10%, at least about 20%, at least about 30%, at least about 40%, at least about 50%, at least about 60%, at least about 70%, at least about 80%, at least about 90%, or 100% of the wells of the multiwell plates contain one or more upstanding wires. At least some of the upstanding wires may be at least partially coated with a biological effector, which can be inserted into cells, as previously discussed.

The multiwell plate format may allow for a variety of insertions to occur in the cells. In some embodiments, relatively large numbers of experiments may be performed. For example, in some cases, commercially-available robotics may be used to add or remove fluids and/or cells to or from at least some of the wells of the multiwell plate and/or to analyze or sense fluids and/or cells in at least some of the wells of the multiwell plate, etc., e.g., allowing for high-throughput experimentation to take place. In one set of embodiments, at least 2, at least 3, at least 5, at least 10, at least 25, at least 50, at least 100, at least 150, at least 200, at least 300, or at least 500 multiwell plates may be operated on by one or more such robotic systems, e.g., to add or remove fluids and/or cells to the multiwell plates.

Non-limiting examples of such robotic systems include liquid handlers that aspirate or dispense liquid samples from and to the multiwell plates, plate movers that can transport multiwell plates between instruments or locations, plate stackers that can store or hold multiwell plates, incubators to control the temperatures that the multiwell plates are exposed to, sensors or plate readers (e.g., ELISA readers) to determine or analyze one or more wells on a multiwell plate, or the like.

Any suitable type of cell may be studied. For example, the cell may be a prokaryotic cell or a eukaryotic cell. The cell may be from a single-celled organism or a multi-celled organism. In some cases, the cell is genetically engineered, e.g., the cell may be a chimeric cell. The cell may be bacteria, fungi, a plant cell, an animal cell, etc. The cell may be from a human or a non-human animal or mammal. For instance, if the cell is from an animal, the cell may be a cardiac cell, a fibroblast, a keratinocyte, a hepatocyte, a chondrocyte, a neural cell, an osteocyte, an osteoblast, a muscle cell, a blood cell, an endothelial cell, an immune cell (e.g., a T-cell, a B-cell, a macrophage, a neutrophil, a basophil, a mast cell, an eosinophil), etc. In some cases, the cell is a cancer cell.

Thus, for instance, a variety of different cell types may be exposed to a common biological effector in certain embodiments, e.g., to determine the effect of the common biological effector on such cells. For example, the biological effector may be a small molecule, RNA, DNA, a peptide, a protein, or the like. As non-limiting examples, the cell types may be bacteria or other prokaryotes, and the common biological effector may be a suspected drug or antimicrobial agent. In some cases, at least 10, at least 20, at least 30, at least 40, at least 50, at least 60, at least 70, at least 80, at least 90, at least 100 cells, at least 500 cells, at least 1000 cells, at least 5000 cells, at least 10,000 cells, at least 50,000 cells, at least 100,000 cells, etc. may be studied. For example, the different cell types may each be placed into distinct wells of a multiwell plate, and nanowires inserted into the cells placed in each of the wells to insert a common biological effector.

In another set of embodiments, different common biological effectors may be studied, e.g., as applied to a single or clonal population of cells, or to a variety of different cell types such as those discussed above. For instance, the wells of a multiwell plate may contain nanowires, and at least some of the nanowires may be at least partially coated with a variety of biological effectors. For example, at least 10, at least 20, at least 30, at least 40, at least 50, at least 60, at least 70, at least 80, at least 90, at least 100, at least 500, at least 1000, at least 5000, at least 10,000, at least 50,000, at least 100,000, etc. different biological effectors may be studied. In some cases, the biological effectors may be added to the wells and the nanowires using robotic systems such as those discussed herein. Accordingly, cells placed in the wells of the multiwell plate may encounter different biological effectors, as inserted by the nanowires. As a non-limiting example, the different biological effectors may represent a plurality of suspected candidate drugs, and the effects of the various candidate drugs on a given population of cells may be studied to identify or screen drugs of interest.

In addition, it should be noted that in some embodiments, the cells may be cultured on the substrate using any suitable cell culturing technique, e.g., before or after insertion of nanowires. For example, mammalian cells may be cultured at 37° C. under appropriate relative humidities in the presence of appropriate cell media. Thus, for instance, the effect of a candidate drug (or a plurality of candidate drugs) on the effect of a suitable population of cells may be studied.

The following documents are incorporated herein by reference in their entireties: U.S. patent application Ser. No. 13/264,587, filed Oct. 14, 2011, entitled “Molecular Delivery with Nanowires,” by Park, et al., published as U.S. Patent Application Publication No. 2012/0094382 on Apr. 19, 2012; International Patent Application No. PCT/US11/53640, filed Sep. 28, 2011, entitled “Nanowires for Electrophysiological Applications,” by Park, et al., published as WO 2012/050876 on Apr. 19, 2012; International Patent Application No. PCT/US2011/53646, filed Sep. 28, 2011, entitled “Molecular Delivery with Nanowires,” by Park, et al., published as WO 2012/050881 on Apr. 19, 2012; U.S. Provisional Patent Application Ser. No. 61/684,918, filed Aug. 20, 2012, entitled “Use of Nanowires for Delivering Biological Effectors into Immune Cells,” by Park, et al.; and U.S. Provisional Patent Application Ser. No. 61/692,017, filed Aug. 22, 2012, entitled “Fabrication of Nanowire Arrays,” by Park, et al. In addition, the following PCT applications, each filed on Mar. 15, 2013, are incorporated herein by reference in their entireties: “Use of Nanowires for Delivering Biological Effectors into Immune Cells,” by Park, et al.; and “Fabrication of Nanowire Arrays,” by Park, et al. The following examples are intended to illustrate certain embodiments of the present invention, but do not exemplify the full scope of the invention.

### EXAMPLE 1

This example demonstrates the fabrication of a 384-well NW plate in accordance with one embodiment of the invention. Biocompatible glue (e.g., Masterbond EP42HT-2ND-2MED BLACK or EP42HT-2 CLEAR) was applied to the back of a bottomless 384-well plate. A silicon wafer large enough to cover all the wells of the plate, with nanowires pre-fabricated and pre-silanized on one side, was applied to the plate such that the glue met the side of the wafer possessing the wires (i.e., NWs face into the wells). Slight movements were made to gently spread the glue and light pressure was applied to ensure secure attachment.

The glue on the merged NW-well platform was then allowed to cure at room temperature for 48 hours (or for different durations at elevated temperatures, e.g., 100° C. for 1 h). The NW plate was then disinfected by submerging the plate in 70% ethanol for 30 min, washed with ultrapure water, and blown dry.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a bottomless multiwell plate; and
a substrate immobilized to the multiwell plate, the substrate comprising a plurality of upstanding nanowires.

2. The article of claim 1, wherein the multiwell plate is a 384-well plate.

3. The article of claim 1, wherein the multiwell plate is a 1536-well plate.

4. The device of any one of claims 1-3, wherein at least some of the nanowires are silicon nanowires.

5. The device of any one of claims 1-4, wherein at least some of the nanowires are at least partially coated with a biological effector.

6. The device of any one of claims 1-5, wherein the nanowires have an average length of less than about 10 micrometers.

7. The device of any one of claims 1-6, wherein the nanowires have an average diameter of less than about 300 nm.

8. The device of any one of claims 1-7, wherein the nanowires have an average density of less than 10 nanowires per micrometer² (μm²).

9. The device of any one of claims 1-8, further comprising a biocompatible glue immobilizing the multiwell plate and the surface.

10. A method, comprising:
immobilizing a substrate comprising a plurality of upstanding nanowires to a bottomless multiwell plate.

11. The method of claim 10, wherein the multiwell plate is a 384-well plate.

12. The method of claim 10, wherein the multiwell plate is a 1536-well plate.

13. The method of any one of claims 10-12, wherein at least some of the nanowires are at least partially coated with a biological effector.

14. The method of any one of claims 10-13, further comprising placing cells in at least one well of the multiwell plate.

15. The method of claim 14, further comprising culturing the cells within the wells of the multiwell plate.

16. The method of any one of claim 14 or 15, wherein the cells are mammalian cells.

17. The method of any one of claims 14-16, wherein the cells are human cells.

18. The method of any one of claims 14-17, wherein the cells are immune cells.

19. The method of any one of claim 14 or 15, wherein the cells are bacterial cells.

20. A method, comprising:
placing a plurality of cells in a plurality of wells in a multiwell plate, wherein at least one of the wells comprises a plurality of upstanding nanowires.

21. The method of claim 20, wherein the multiwell plate is a 384-well plate.

22. The method of claim 20, wherein the multiwell plate is a 1536-well plate.

23. The method of any one of claims 20-22, wherein at least some of the nanowires are at least partially coated with a biological effector.

24. The method of claim 23, wherein a first well of the multiwell plate comprises first upstanding nanoscale wires at least partially coated with a first biological effector, and a second well of the multiwell plate comprises second nanoscale wires at least partially coated with a second biological effector different from the first biological effector.

25. The method of any one of claims 20-24, comprising placing a first plurality of cells in a first well of the multiwell plate, and placing a second plurality of cells in a second well of the multiwell plate.

26. A method, comprising:
placing at least 10 distinct cell types into at least 10 distinct wells of a multiwell plate; and
inserting a plurality of nanowires coated with a common biological effector into each of the at least 10 distinct cell types.

27. The method of claim 26, comprising placing at least 100 distinct cell types into at least 100 distinct wells of a multiwell plate, and inserting a plurality of nanowires coated with an identical biological effector into each of the at least 100 distinct cell types.

28. A method, comprising:
placing cells into at least 10 distinct wells of a multiwell plate; and
inserting a plurality of nanowires into the cells, at least some of the nanowires at least partially coated with a biological effector, wherein in each of the 10 distinct wells, a different biological effector is inserted into the cells in the respective wells.

29. The method of claim 28, comprising placing cells into at least 100 distinct wells of a multiwell plate, and inserting a plurality of nanowires into the cells, at least some of the nanowires at least partially coated with a biological effector, wherein in each of the 100 distinct wells, a different biological effector is inserted into the cells in the respective wells.

30. The method of any of claim 28 or 29, wherein more than one type of cell is inserted in at least one of the wells.
